(19) United States
(12) Patent Application Publication
(10) Pub. No.: US 2013/0110234 A1
(43) Pub. Date: May 2, 2013

(54) DUAL OPTIC ACCOMMODATING IOL WITH LOW REFRACTIVE INDEX GAP MATERIAL

(71) Applicants: Lauren DeVita, Fort Worth, TX (US); Hari Subramamiam, Irvine, CA (US); Shinwook Lee, Arlington, TX (US); Daniel Robert Carson, Fort Worth, TX (US); David Borja, Fort Worth, TX (US); Son Tran, Arlington, TX (US); Kevin Mark Lewellen, Arlington, TX (US)

(72) Inventors: Lauren DeVita, Fort Worth, TX (US); Hari Subramamiam, Irvine, CA (US); Shinwook Lee, Arlington, TX (US); Daniel Robert Carson, Fort Worth, TX (US); David Borja, Fort Worth, TX (US); Son Tran, Arlington, TX (US); Kevin Mark Lewellen, Arlington, TX (US)

(21) Appl. No.: 13/617,488

(22) Filed: Sep. 14, 2012

Related U.S. Application Data
(60) Provisional application No. 61/552,869, filed on Oct. 28, 2011.

(51) Int. Cl. A61F 2/16

(52) U.S. Cl. 623/6.34

(57) ABSTRACT

An accommodative intraocular lens (IOL) system is disclosed for insertion into an eye to provide accommodative vision, the system including a first lens having an first optic, a second lens having a second optic, a transparent, low refractive index medium disposed between the first and second optics; and at least one haptic connected to the first and second lenses and configured to facilitate movement of one lens relative to the other lens, such that when the lens system is positioned in an eye, ciliary muscle movements can alter the distance between the first and second lenses and vary the overall lens power of the system.

### RELATED APPLICATIONS

This application claims priority to U.S. provisional application Ser. No. 61/552,869, filed on Oct. 28, 2011, the contents which are incorporated herein by reference.

### TECHNICAL FIELD

This invention relates generally to the field of intraocular lenses (IOL) and, more particularly, to accommodative IOLs.

### BACKGROUND OF THE INVENTION

The human eye in its simplest terms functions to provide vision by transmitting light through a clear outer portion called the cornea, and focusing the image by way of a crystalline lens onto a retina. The quality of the focused image depends on many factors including the size and shape of the eye, and the transparency of the cornea and the lens.

When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an artificial intraocular lens (IOL).

Cataractous lenses may be removed by a surgical technique called phacoemulsification. During this procedure, an opening is made in the anterior capsule and a thin phacoemulsification cutting tip is inserted into the diseased lens and vibrated ultrasonically. The vibrating cutting tip liquifies or emulsifies the lens so that the lens may be aspirated out of the eye. The diseased lens, once removed, is replaced by an artificial lens.

In the natural lens, bifocality of distance and near vision is provided by a mechanism known as accommodation. The natural lens, early in life, is soft and contained within the capsular bag. The bag is suspended from the ciliary muscle by the zonules. Relaxation of the ciliary muscle tightens the zonules, and stretches the capsular bag. As a result, the natural lens tends to flatten. Tightening of the ciliary muscle relaxes the tension on the zonules, allowing the capsular bag and the natural lens to assume a more rounded shape. In this way, the natural lens can be focus alternatively on near and far objects.

As the lens ages, it becomes harder and is less able to change shape in reaction to the tightening of the ciliary muscle. This makes it harder for the lens to focus on near objects, a medical condition known as presbyopia. Presbyopia affects nearly all adults over the age of 45-50.

Prior to the present invention, when a cataract or other disease required the removal of the natural lens and replacement with an artificial IOL, the IOL typically was a monofocal lens, requiring that the patient use a pair of spectacles or contact lenses for near vision.

There have been some attempts to make a two-optic accommodative lens system. For example, U.S. Pat. No. 5,275,623 (Sarfarazi), WIPO Publication No. 00/66037 (Glick, et al.) and WO 01/34067 A1 (Bandhauer, et al), the entire contents of which are incorporated herein by reference, all disclose a two-optic lens system with one optic having a positive power and the other optic having a negative power. The optics are connected by a hinge mechanism that reacts to movement of the ciliary muscle to move the optics closer together or further apart, thereby providing accommodation.

Prior art accommodative two lens systems using a movable “zoom” lens have inherently limited movement. The maximum sensitivity or movement magnification (a unitless ratio) is defined as the axial movement of the lens per unit zonule movement and is derived by the following equation:

a=−B/A

where B is the projected distance of the zonule length which is in the order of 1.0 to 2.0 mm; and A is the axial distance between the middle plane between the dual lens and the anterior surface of the anterior lens where the zonules terminate.

U.S. Patent Application Pub. No. US2007/0050024, the entire contents of which are incorporated herein by reference, discloses the use of a cam mechanism to increase the range of relative movement between the elements of a two-optic system.

However, even with a cam element or other mechanism for increasing the range of movement in dual optic systems, it is difficult to obtain an accommodative amplitude that would restore the normal accommodation of a healthy eye, e.g., a power shift on the order of 4 diopters, due to the refractive limitations of conventional lens materials and the limited space available within the capsule. Consequently, patients can have refractive errors after the implantation of the IOL and still need additional spectacles corrections that are not desired.

Accordingly, there exists a need for better solutions to the problem of accommodation in IOLs. In particular, dual optic accommodative lens that could provide greater accommodative amplitude would satisfy a long-felt need in the field.

### SUMMARY OF THE INVENTION

To overcome the above and other drawbacks of conventional systems, the present invention provides an intraocular lens system for insertion into an eye to provide accommodative vision, the system including a first lens having an first optic, a second lens having a second optic, a transparent, low refractive index medium disposed between the first and second optics; and at least one haptic connected to the first and second lenses and configured to facilitate movement of one optic relative to the other optic, such that when the lens system is positioned in an eye, ciliary muscle movements can alter the distance between the first and second lenses and vary the overall lens power of the system.

To enhance the accommodative effect of the relative movement of the first and second optics, the transparent medium should have an index of refraction less than that of the aqueous humor, e.g., less than about 1.34, more preferably less than about 1.1 in order to provide a greater range of accommodation.

In certain embodiments, the transparent medium can be a gas, such as air. In some instances, it can be useful to use an inert gas such as argon, which also has a lower permeability vis-à-vis a sealing enclosure due, at least in part, to its higher molecular weight. Thus, the transparent medium, for example, can be composed by weight (or by volume) of at least 80% or 85%, or 90% or 95% or even 98% percent or higher of argon gas. In other applications, other fluids, e.g., liquids or gases, can be used so long as the index of refraction is lower than that of the lens elements and/or the ambient ocular environment.

In certain embodiments, the first lens can be an anterior lens (closest to the cornea or front of the eye), which includes a high positive power optic while the second lens can be a posterior lens (closest to the retina or back of the eye), which includes a negative optic such that relative movement of the anterior and posterior optics changes the overall power of the lens system.

The haptic can join the first and second lenses (or optics) together via a flexible hinge. The haptic (or the overall system) can further include a sealing enclosure for the transparent medium. Moreover, the haptic or system can further include force amplifying elements, such one or more lever arms that translate the forces applied by the ciliary muscle into relative movement of one or more of the optics along the optical axis of the lens system to provide as desired level of accommodation, e.g., preferably at least about 3 diopters, or more preferably at least about 4 diopters in an eye.

In another aspect of the invention, methods of restoring accommodation in an eye are disclosed in which an intraocular lens system is provided having a first lens having an first optic, a second lens having a second optic; a transparent, low refractive index medium disposed between the first and second optics, and at least one haptic connected to the first and second lenses and configured to facilitate movement of one lens relative to the other lens. The methods include a step of positioning the lens system in an eye in a manner whereby changes in a ciliary muscle will be transmitted to the system such that ciliary muscle movements alter the distance between the first and second lenses and vary the overall lens power of the system.

In yet another aspect of the invention, methods of manufacturing accommodative intraocular lens systems are disclosed by providing a first lens having an first optic, providing a second lens having a second optic and disposing a transparent, low refractive index medium between the first and second optics The manufacturing method can further include the step of joining the first and second lenses together with a flexible haptic configured to facilitate movement of one lens relative to the other lens, whereby when the lens system is positioned in an eye, changes in the position of the ciliary muscle will be transmitted to the system such that ciliary muscle movements alter the distance between the first and second lenses and vary the overall lens power of the system.

### BRIEF DESCRIPTION OF THE DRAWINGS

The invention will be more fully understood from the following detailed description taken in conjunction with the accompanying drawings, in which:

FIG. 1A is an perspective schematic illustration of a dual optic accommodative lens system according to the invention;

FIG. 1B is a perspective schematic illustration of the dual optic accommodative lens system of FIG. 1A in a second configuration according to the invention;

FIG. 2A is a cross-sectional schematic illustration of the lens system configuration of FIG. 1A;

FIG. 2B is a cross-sectional schematic illustration of the lens system configuration of FIG. 1B;

FIG. 3A is a perspective schematic illustration of another embodiment of dual optic accommodative lens system according to the invention;

FIG. 3B is a perspective schematic illustration of the dual optic accommodative lens system of FIG. 3A in a second configuration according to the invention;

FIG. 4A is a cross-sectional schematic illustration of the lens system configuration of FIG. 3A;

FIG. 4B is a cross-sectional schematic illustration of the lens system configuration of FIG. 3B;

FIG. 5A is a perspective schematic illustration of yet another embodiment of a dual optic accommodative lens system according to the invention;

FIG. 5B is a perspective schematic illustration of the dual optic accommodative lens system of FIG. 5A in a second configuration according to the invention;

FIG. 6A is a cross-sectional schematic illustration of the lens system configuration of FIG. 5A;

FIG. 6B is a cross-sectional schematic illustration of the lens system configuration of FIG. 5B;

FIG. 7A is cross-sectional schematic side view of dual optic accommodative lens system with force-transmitting ring and haptic assembly in a low power or distance vision state;

FIG. 7B is cross-sectional schematic side view of dual optic accommodative lens system with force-transmitting ring and haptic assembly in medium power or intermediate vision state;

FIG. 7C is cross-sectional schematic side view of dual optic accommodative lens system with force-transmitting ring and haptic assembly in a high power or near vision state; and

FIG. 8 is a graph of accommodation (in diopters) versus lens separation (in mm) for optics separated by air as compared to the same separation by water.

### DETAILED DESCRIPTION

Certain exemplary embodiments will now be described to provide an overall understanding of the principles of the methods and devices disclosed herein. One or more examples of these embodiments are illustrated in the accompanying drawings. Those skilled in the art will understand that the methods and devices specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

One class of accommodating IOLs (AIOLs) currently under development is often referred to as “dual-optic.” Such systems utilize two lenses of high refractive index (relative to aqueous humor). Typically, the anterior lens is a high power lens designed to move anteriorly in the eye when a patient focuses on near objects. The posterior lens is usually a negative lens and sometimes moves in response to the accommodation apparatus as well. The space between these lenses becomes filled with aqueous humor. The setup of this system has an inherent limitation of accommodation amplitude due to the small space available in the eye.

Filling the gap between the lenses with air or other low refractive index gas, liquid or gel offers a simple method to overcoming this limitation. The accommodation amplitude for the same lens displacement is increased based on the difference between the refractive index of the gap and aqueous humor. For air, the potential accommodation amplitude can be increased by a factor of about 3 when an AIOL according to the present invention is implanted in an eye.

The invention uses a low index of refraction material to fill the gap between two high index of refraction lenses to form an accommodating lens system. This can be achieved in a number of ways: the two lenses can be connected in the equator 360 degrees to seal the gap; the two lenses can be coupled by a flexible balloon filled with air or other low index of refraction material; the two lenses can each have a flexible or non-flexible additional layer with a low index of refraction material that mimics the effect of completely filling the gap. The optical portion of the system is coupled to the eye via haptics. The system responds to the normal accommodation apparatus and can be linked directly or indirectly the contraction and relaxation of the ciliary muscle.

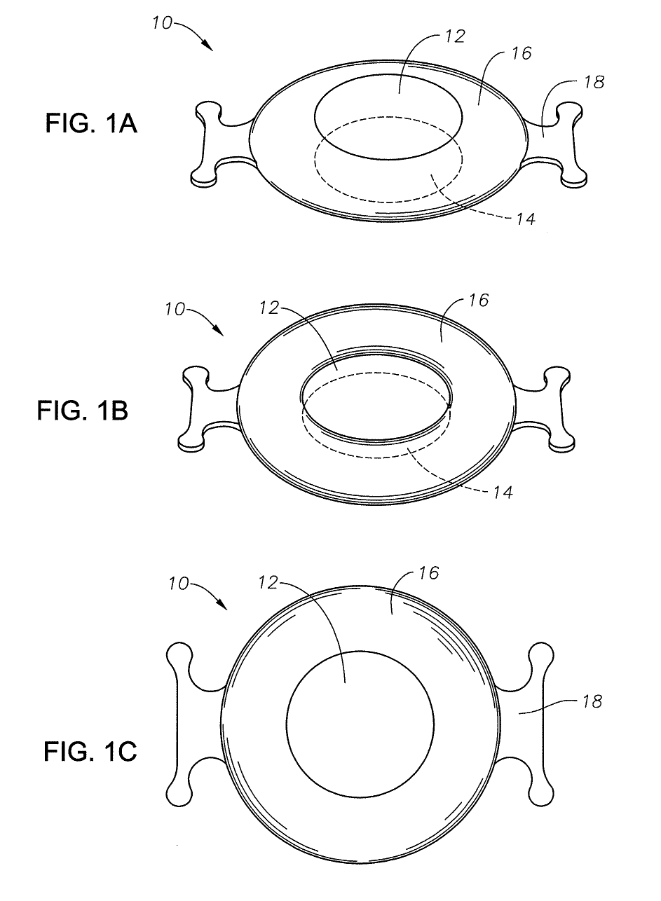

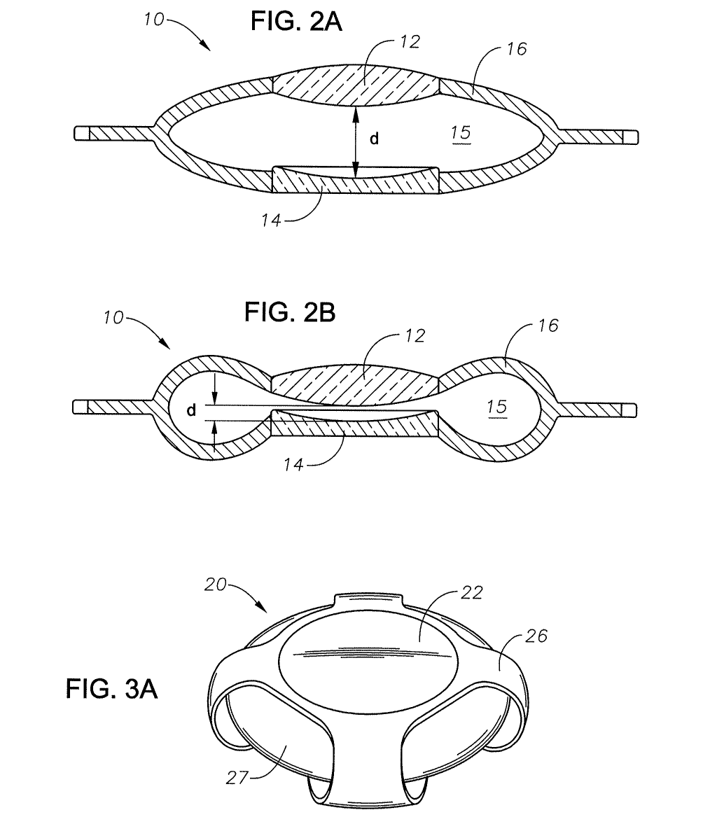

FIGS. 1A-1C and 2A-2B provide a schematic illustration of one such dual optic system with a gap between the lens elements filled with a low index of refraction material. In these figures, an accommodating IOL 10 is shown having a first optic 12 and a second optic 14. The optics 12, 14 are joined to a flexible haptic 16, which may optionally have projections 18 for alignment or engagement within the lens capsule (shown in phantom in FIGS. 2A and 2B). In response to movement of the ciliary muscle, the flexible haptic is adapted to change shape (as shown in FIGS. 1 C and 2B) such that the air gap between the optics is reduced.

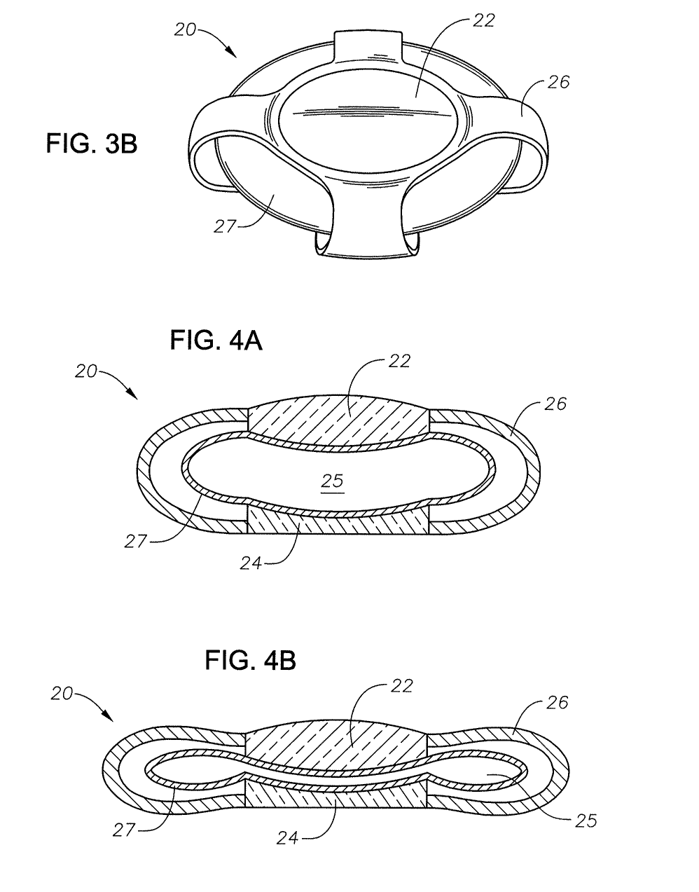

FIGS. 3A-3B and 4A-4B illustrate a second embodiment of a dual optic system 20 according to the invention again having a first optic 22 and a second optic 24. The optics 22, 24 are similarly joined to a flexible haptic 26. However, in this embodiment, a separate flexible chamber 27 filled with air or a similar low refractive index fluid is disposed between the first and second optics. In response to movement of the ciliary muscle, the flexible haptic and flexible chamber are adapted to change shape (as shown in FIG. 4B) such that the air gap between the optics is reduced.

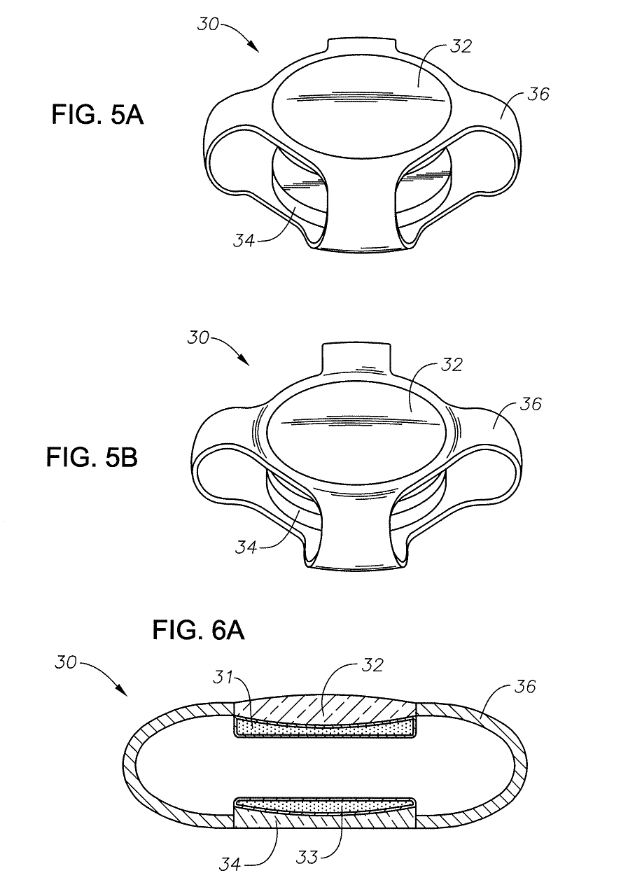

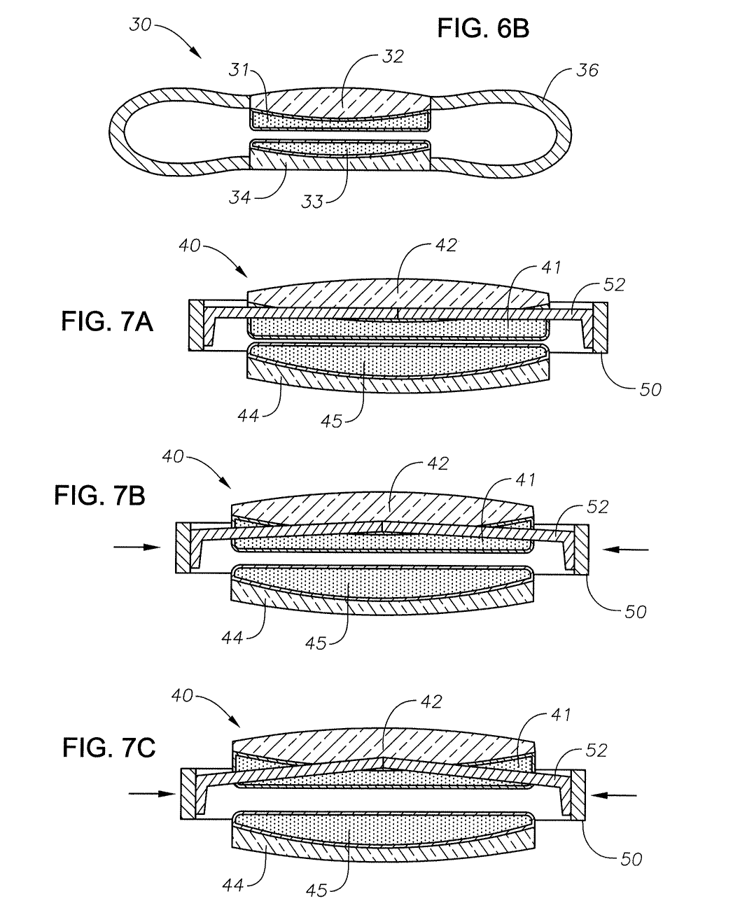

FIGS. 5A-3B and 6A-4B illustrate a third embodiment of a dual optic system 30 according to the invention again having a first optic 32 and a second optic 34. The optics 32, 34 are again joined to a flexible haptic 36. However, in this embodiment, optic 32 is joined to a first low refractive index chamber 31, e.g., a rigid or flexible shell again filled with air or a similar low refractive index fluid and, optionally, optic 34 is likewise joined to a first low refractive index chamber 33, e.g., again a rigid or flexible shell again filled with air or a similar low refractive index fluid. (It should be clear that a low refractive index optical element can be joined to either the optic 32 or the optic 34 or both and desired effect of amplifying accommodation will be achieved so long as the low refractive index optical element occupies at least a portion of the space between optics 32 and 34). Again, in response to movement of the ciliary muscle, the flexible haptic is adapted to change shape (as shown in FIGS. 6A and 6B) such that the gap between the optics is reduced.

Various techniques are known to those skilled in the art to transform the movements of ciliary muscles into relative motion of optics in dual optic systems. FIGS. 7A-7C illustrate one such dual optic accommodative lens system with a force-transmitting ring and haptic assembly 40. The force transmitting ring and haptic assembly 40 includes hinged haptics 52 attached to first haptic 42 and a ring 50 joined to second optics 44. The ring is further configured to receive the hinge haptics and exert radial pressure thereon in response to ciliary muscle movements. In a manner similar to the third embodiment discussed above, optic 42 can be joined to a first low refractive index chamber 41, e.g., a rigid or flexible shell again filled with air or a similar low refractive index fluid and, optionally, optic 44 can likewise be joined to a first low refractive index chamber 45, e.g., again a rigid or flexible shell again filled with air or a similar low refractive index fluid. (It should be clear that the first or second embodiment can likewise be implemented with the force transmitting ring as well.) The inward radial pressure exerted by ring 50 causes the hinged haptic 52 to bend (as shown progressively in FIGS. 7B and 7C) and urge the first optic 42 upward (e.g., in an anterior direction when placed in the eye.) For further details on force transmitting systems for accommodative IOLs, see US Published Pat. Appl. No. US 2007/0050024 by Zhang, herein incorporated in its entirety by reference.

To demonstrate the invention, PMMA prototypes were fabricated. High power anterior lenses (Radius of curvature 1=8.72 mm, Radius of curvature 2=−8.72 mm, edge thickness=1.5 mm, optic diameter=6.0 mm) were attached to negative lenses (Radius of curvature 1=−8.72 mm, Radius of curvature 2=−41.58 mm, edge thickness=1.5 mm, optic diameter=6.0 mm) using 3M VHB 4905 (0.5 mm thick adhesive tape). Gaps between lenses were approximately 0.5 mm and 1.5 mm (achieved by using a single layer of VHB and 3 layers of VHB respectively). One set of lenses was completely sealed around the equator to keep air in and water out. The other set of lenses was filled with water. Measurements of the lens systems corresponded well to calculated optical power change:

TABLE 1
Measured v. Predicted Power

| | Measured Power | Predicted Power |
|---|---|---|
| | Change (D/mm) | Change (D/mm) |
| Air Gap | 5.42 | 5.27 |
| Water | 0.54 | 0.49 |
| Gap | | |

### Optical Simulation

The optical performance of the proposed dual-optic AIOL and a conventional dual-optic AIOL were evaluated in ray tracing software. The optical performance in terms of accommodative efficiency in units of [D/mm] is the dioptric change in near focus as a result of AIOL lens movement. The evaluation was performed in the Alcon-Navarro eye model with Zemax ray tracing software.

TABLE 2
Assumptions for Optical Simulation

| | Radius of | | | | |
|---|---|---|---|---|---|
| | Curvature | Thickness | Refractive | Aperture | |
| Surface | (mm) | (mm) | Index | (mm) | Conic |
| Object | Infinity | Infinity* | 1 | | |
| Spectacle Plane | Infinity | 5 mm | 1 | | |
| Ant Cornea | 7.72 | 0.55 | 1.376 | 8 | −0.183 |
| Post Cornea | 6.50 | | | | |
| Aqueous | | 3.05 | 1.336 | | |
| Iris | | | | 6 | |
| Aqueous | | 2.00* | | | |
| Front IOL 1 | 11.93 | 0.88 | 1.548 | 6 | |
| Front IOL 2 | −12.15 | | | | |
| Air Gap or | | 0.7* | V* | | |
| Aqueous* | | | | | |
| Back IOL 1 | −7.41 | 0.24 | 1.548 | 6 | |
| Back IOL 2 | −15.39 | | | | |
| Vitreous | | V* | 1.336 | | |

V* variables for optimization.
*iteratively adjusted variables and initial values.

The system was initially optimized by adjusting the vitreous chamber length until an object at infinity produced a minimum spot size. The front lens first surface was placed 2 mm posterior to the iris. Accommodation was modeled by an anterior movement of the front lens in 0.1 mm increments to a maximum of 1 mm and an increase in separation between the front and back lens from 0.7 to 1.7 mm.

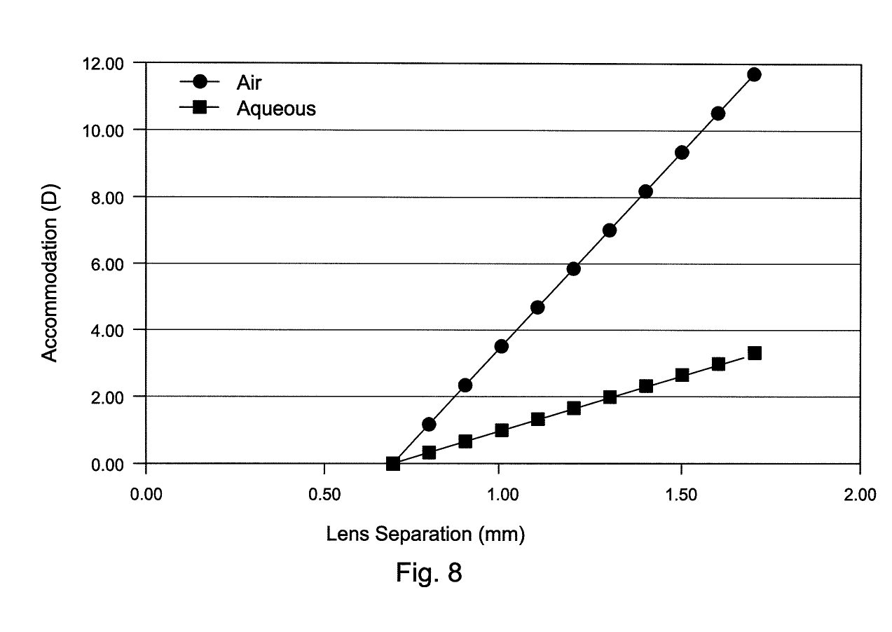

In FIG. 8 the results of the optical simulation are presented in graphic form. As shown in FIG. 8, the conventional dual optic AIOL had an accommodative efficiency of 3.2 D/mm while the accommodative efficiency of the dual optic AIOL with air spacing increased to 11.62 D/mm.

All of the embodiments described above are non-limiting examples of the present invention only. In addition, all papers and publications cited herein are hereby incorporated by reference in their entirety. One of skill in the art will appreciate further features and advantages of the invention based on the above-described embodiments. Accordingly, the invention is not to be limited by what has been particularly shown and described, except as indicated by the appended claims.

What is claimed is:

1. An intraocular lens system for insertion into an eye to provide accommodative vision, the system comprising:
a first lens having a first optic;
a second lens having a second optic;
a transparent, low refractive index medium disposed between the first and second optics; and
at least one haptic connected to the first and second lenses and configured to facilitate movement of one optic relative to the other optic, such that when the lens system is positioned in an eye, ciliary muscle movements can alter the distance between the first and second optics and vary the overall lens power of the system.

2. The lens system of claim 1, wherein the transparent medium has an index of refraction less than 1.34

3. The lens system of claim 1, wherein the transparent medium has an index of refraction less than 1.1

4. The lens system of claim 1, wherein the transparent medium comprises a gas.

5. The lens system of claim 1, wherein the transparent medium comprises air.

6. The lens system of claim 1, wherein the transparent medium comprises argon.

7. The lens system of claim 1, wherein the transparent medium comprises at least 90 percent argon.

8. The lens system of claim 1, wherein the first lens is an anterior positive lens.

9. The lens system of claim 1, wherein the second lens is a posterior negative lens.

10. The lens system of claim 1, wherein the haptic further comprises a lever joined to at least one of the optics by a hinge.

11. The lens system of claim 1 wherein the haptic comprises a flexible V-shaped lever.

12. The lens system of claim 1, wherein the haptic further comprises a force transmitting ring.

13. The lens system of claim 1, wherein the haptic surrounds the transparent, low index medium and provides a sealing enclosure for the medium.

14. The lens system of claim 1, wherein the transparent, low index medium is contained by a sealing enclosure separate from the haptic.

15. The lens system of claim 1, wherein the transparent, low index medium is contained by a sealing enclosure separate from at least one of the optics.

16. The lens system of claim 1, wherein the transparent, low index medium is contained by a sealing enclosure separate from both the first optic and the second optic.

17. The lens system of claim 1, wherein the transparent, low index medium is contained by a sealing enclosure joined to at least one of the optics.

18. The lens system of claim 1, wherein the transparent, low index medium is contained in two separate sealing enclosures, each joined to one or the other of the optics.

19. The lens system of claim 1, wherein a range of haptic displacement provides an accommodation of at least about 3 diopters in an eye.

20. The lens system of claim 1, wherein a range of haptic displacement provides an accommodation of at least about 4 diopters in an eye.

21. A method of restoring accommodation in an eye, the method comprising
providing an intraocular lens system having a first lens having an first optic, a second lens having a second optic; a transparent, low refractive index medium disposed between the first and second optics, and at least one haptic connected to the first and second lenses and configured to facilitate movement of one lens relative to the other lens; and
positioning the lens system in an eye in a manner whereby changes in a ciliary muscle will be transmitted to the system such that ciliary muscle movements alter the distance between the first and second lenses and vary the overall lens power of the system.

22. A method of manufacturing an accommodative intraocular lens system, the method comprising
providing a first lens having an first optic,
providing a second lens having a second optic;
disposing a transparent, low refractive index medium between the first and second optics, and
joining the first and second lenses together with a flexible haptic configured to facilitate movement of one lens relative to the other lens;
whereby when the lens system is positioned in an eye, changes in a ciliary muscle will be transmitted to the system such that ciliary muscle movements alter the distance between the first and second lenses and vary the overall lens power of the system.
